(19) United States
(12) Patent Application Publication
(10) Pub. No.: US 2014/0257077 A1
(43) Pub. Date: Sep. 11, 2014

(54) IMAGING DEVICES AND METHODS OF USE THEREOF

(71) Applicant: VOLCANO CORPORATION, San Diego, CA (US)

(72) Inventor: Bret Millett, Folsom, CA (US)

(73) Assignee: VOLCANO CORPORATION, San Diego, CA (US)

(21) Appl. No.: 14/200,943

(22) Filed: Mar. 7, 2014

Related U.S. Application Data
(60) Provisional application No. 61/774,875, filed on Mar. 8, 2013.

(51) Int. Cl. A61B 5/00

(52) U.S. Cl. CPC A61B 5/0095
USPC 600/407

(57) ABSTRACT

The invention generally relates to imaging devices and methods of use thereof. Aspects of the invention provide an imaging device that includes a core wire and more than 32 optical fibers coupled to the core wire.

### RELATED APPLICATION

The present application claims the benefit of and priority to U.S. provisional patent application Ser. No. 61/774,875, filed Mar. 8, 2013, the content of which is incorporated by reference herein in its entirety.

### FIELD OF THE INVENTION

The invention generally relates to imaging devices and methods of use thereof.

### BACKGROUND

Biomedical imaging technology is rapidly advancing. For example, magnetic resonance imaging (MRI), X-ray computed tomography, and confocal microscopy are all in widespread research and clinical use, and have resulted in fundamental and dramatic improvements in health care. However, there are many situations in which existing biomedical imaging technologies are not adequate. This is particularly true where high resolution (e.g. approximately 5-10 μm) imaging is required. In these situations, such imaging technology does not provide a physician with the required diagnostic information, and the physician must resort to other invasive examinations, such as biopsy and histopathologic examination, in order to obtain the required diagnostic information. Such examinations are potentially harmful, time consuming, and costly. Furthermore, there are many situations in which conventional excisional biopsy is not possible.

Imaging technologies have been developed that addresses those concerns. For example, Intravascular Ultrasound (IVUS) is an important interventional diagnostic procedure for imaging atherosclerosis and other vessel diseases and defects. Development of depth-resolved light reflection or Optical Coherence Tomography (OCT) provides a high resolution imaging technique for analyzing tissue. OCT is an imaging technique that captures micrometer-resolution, three-dimensional images from within optical scattering media (e.g., biological tissue). More recently, guidewires have been equipped with imaging capabilities.

### SUMMARY

The invention recognizes that different parameters are required to image different types of vessels, e.g., coronary vessels versus peripheral vessels and other larger structures (e.g., greater than 5 mm). For example, coronary imaging guidewires are designed to image within coronary vessels. Coronary vessels typically have luminal diameters of less than 5 mm. Accordingly, coronary imaging guidewires are significantly less than 5 mm in diameter. Having such a small diameter limits the number of optical fibers that can be associated with such imaging guidewires.

For optimal detection using an imaging guidewire, optical fibers associated with the guidewire need to be within close proximity (e.g., about or less than 2 mm) from a vessel wall to produce a high resolution image. Accordingly, such coronary imaging guidewires are insufficient for imaging larger structures, such as the esophageal walls (luminal diameter of about 18 mm to 24 mm) or the gastrointestinal walls (luminal diameter of about 2 cm to about 10 cm), due to distance from the vessel walls or inadequate imaging power.

The invention provides imaging devices for imaging larger structures. Devices of the invention include a core wire that is large enough so that the imaging elements of the device are close enough to the walls (e.g., about or within 2 mm) of a larger structure to produce a high resolution image. Increasing the diameter of the core wire, also increases the surface area of the wire, allowing a larger number of optical fibers to be coupled to the core wire, thereby increasing the imaging power of the device over that of coronary imaging guidewires. Typically, imaging devices of the invention will use more than 32 optical fibers, e.g., 34, 36, 38, 40, 64, 96, 128, etc. In this manner, an imaging device is provided that positions the optical fibers close to the wall of a larger structure and also provides enough imaging power to produce a high resolution image of that structure.

In certain embodiments, the core wire is sized to image the esophagus, and thus will have a diameter of from about 18 mm to about 24 mm. In other embodiments, the core wire is sized to image the gastrointestinal walls, and thus will have a diameter from about 2 cm to about 10 cm, depending on which part of the gastrointestinal system is to be imaged.

Typically, the optical fibers will surround the core wire, although this is not required. Semi-circular and other configurations are within the scope of the invention. In certain embodiments, the optical fiber includes at least one opto-acoustic sensor. Generally, the opto-acoustic sensor will include an optical fiber having a blazed fiber Bragg grating, a light source that transmits light through the optical fiber, and a photoacoustic transducer material positioned so that it receives light diffracted by the blazed fiber Bragg grating and emits ultrasonic imaging energy.

Another aspect of the invention provides methods for analyzing tissue surrounding a lumen. Those methods involve providing an imaging device, the device having a core wire and more than 32 optical fibers coupled to the core wire. The method additionally involves inserting the device into a body lumen, using the device to image tissue that surrounds the body lumen, and analyzing an image of the tissue, thereby analyzing the tissue surrounding the lumen.

### BRIEF DESCRIPTION OF THE DRAWINGS

FIG. 1 is a schematic diagram of a conventional optical fiber.

FIG. 2 is a cross-sectional schematic diagram illustrating generally one example of a distal portion of an imaging device that combines an acousto-optic Fiber Bragg Grating (FBG) sensor with an photoacoustic transducer.

FIG. 3 is a schematic diagram of a Fiber Bragg Grating based sensor

FIG. 4 is a cross-sectional schematic diagram illustrating generally one example of the operation of a blazed grating FBG photoacoustic transducer.

FIG. 5 is a schematic diagram that illustrates generally one such phased array example, in which the signal to/from each array transducer is combined with the signals from the other transducers to synthesize a radial image line.

FIG. 6 is a schematic diagram that illustrates generally an example of a side view of a distal portion of a device.

FIG. 7 is a schematic diagram that illustrates generally one example of a cross-sectional side view of a distal portion of a device.

FIG. 8 is a block diagram illustrating generally one example of the imaging device and associated interface components.

FIG. 9 is a block diagram illustrating generally another example of the imaging device and associated interface components, including tissue characterization and image enhancement modules.

### DETAILED DESCRIPTION

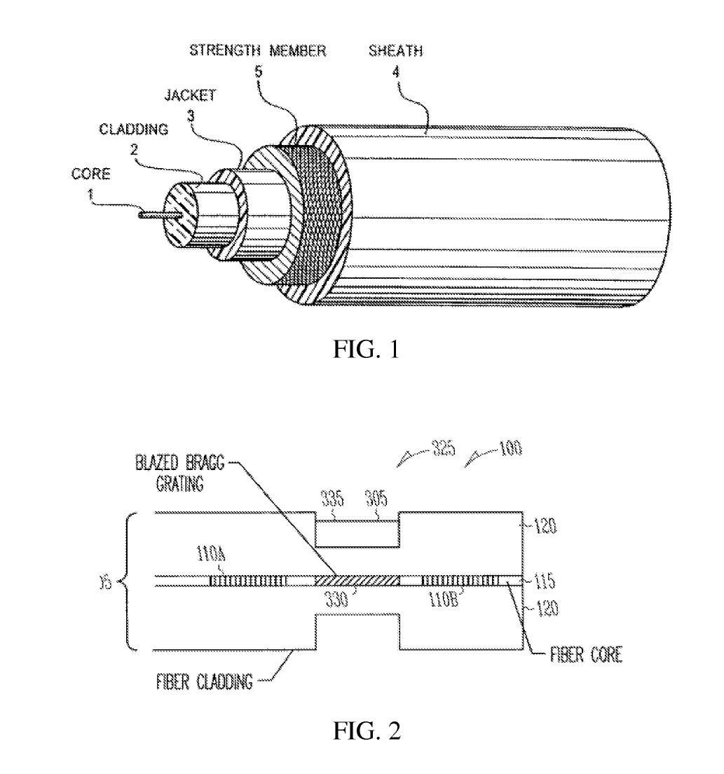

The invention generally relates to imaging devices and methods of use thereof. An exemplary device is shown in FIG. 1. The imaging device includes a core wire and more than 32 optical fibers coupled to the core wire. Devices of the invention are configured for imaging larger structures, such as esophageal and gastrointestinal walls, as opposed to coronary vessels. To that end, the core wire 1000 of devices of the invention typically has a diameter greater than 5 mm. For example, 6 mm, 7 mm, 8 mm, 9 mm, 10 mm, 20 mmm, 30 mm, 40 mm, 50 mm, 75 mm, 1 cm, 2 cm, 3 cm, 4 cm, 5 cm, 6 cm, 7 cm, 8 cm, 9 cm, 10 cm, 15 cm, 20 cm, etc. Additionally, devices of the invention use greater than 32 optical fibers 1001. For example, devices of the invention can use 33 fibers, 34 fibers, 35 fibers, 36 fibers, 40 fibers, 50 fibers, 60 fibers, 64 fibers, 96 fibers, 128 fibers, etc. Using larger diameter core wires places the optical fibers with proximity (e.g., within 5 mm, 4 mm, 3 mm, 2 mm, 1 mm, less than one mm) to the wall of the larger structures. Using greater than 32 optical fibers provides the imaging power necessary for obtaining a high resolution image of the walls of the larger structures.

The imaging device may include a flexible atraumatic distal tip coupled to the core wire. For example, an integrated distal tip can increase the safety of the imaging device by providing a smoother inner diameter for ease of tissue movement. During manufacturing, the transition from the core wire to the flexible distal tip can be finished with a polymer laminate over a distal end of the core wire. No weld, crimp, or screw joint is usually required. The atraumatic distal tip permits advancing the imaging device distally through the blood vessel or other body lumen while reducing any damage caused to the body lumen by the imaging device. In some exemplary configurations, the atraumatic distal tip includes a coil. In some configurations the distal tip has a rounded, blunt distal end.

In certain embodiments, the optical fibers completely surround the core wire. In other embodiments, the optical fibers only partially surround the core wire, such as in a semi-circular configuration. The invention is not limited to any particular configuration of the optical fibers to the core wire.

A plurality of the optical fibers include an optical-acoustic sensor. In certain embodiments, each optical fiber includes an optical-acoustic sensor. Exemplary optical-acoustic imaging sensors are shown for example in, U.S. Pat. No. 7,245,789; U.S. Pat. Nos. 7,447,388; 7,660,492; U.S. Pat. No. 8,059,923; US 2012/0108943; and US 2010/0087732, the content of each of which is incorporated by reference herein in its entirety. Additional optical-acoustic sensors are shown for example in U.S. Pat. No. 6,659,957; U.S. Pat. No. 7,527,594; and US 2008/0119739, the content of each of which is incorporated by reference herein in its entirety.

An exemplary optical-acoustic imaging sensor includes a photoacoustic transducer and a blazed Fiber Bragg grating. Optical energy of a specific wavelength travels down a fiber core of optical fiber and is reflected out of the optical fiber by the blazed grating. The outwardly reflected optical energy impinges on the photoacoustic material. The photoacoustic material then generates a responsive acoustic impulse that radiates away from the photoacoustic material toward nearby biological or other material to be imaged. Acoustic energy of a specific frequency is generated by optically irradiating the photoacoustic material at a pulse rate equal to the desired acoustic frequency.

The optical-acoustic imaging sensor utilizes at least one and generally more than one optical fiber, for example but not limited to a glass fiber at least partly composed of silicon dioxide. The basic structure of a generic optical fiber is illustrated in FIG. 1, which fiber generally consists of layered glass cylinders. There is a central cylinder called the core 1. Surrounding this is a cylindrical shell of glass, possibly multilayered, called the cladding 2. This cylinder is surrounded by some form of protective jacket 3, usually of plastic (such as acrylate). For protection from the environment and more mechanical strength than jackets alone provide, fibers are commonly incorporated into cables. Typical cables have a polyethylene sheath 4 that encases the fibers within a strength member 5 such as steel or Kevlar strands.

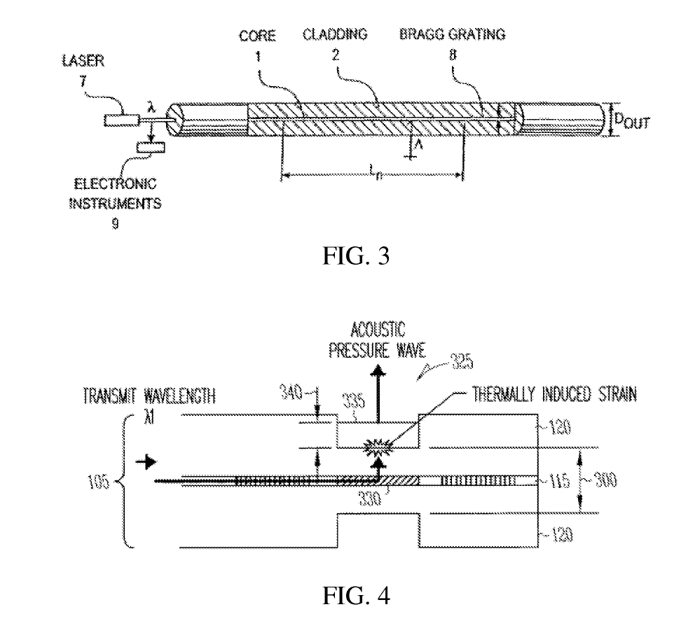

FIG. 2 is a cross-sectional schematic diagram illustrating generally one example of an acousto-optic Fiber Bragg Grating (FBG) sensor 100 with a photoacoustic transducer 325. The optical fiber includes a blazed Fiber Bragg grating. Fiber Bragg Gratings form an integral part of the optical fiber structure and can be written intracore during manufacture or after manufacture. As illustrated in FIG. 3, when illuminated by a broadband light laser 7, a uniform pitch Fiber Bragg Grating element 8 will reflect back a narrowband component centered about the Bragg wavelength λ given by λ=2nλ, where n is the index of the core of the fiber and λ represents the grating period. Using a tunable laser 7 and different grating periods (each period is approximately 0.5 μm) situated in different positions on the fiber, it is possible to make independent measurement in each of the grating positions.

Referring back to FIG. 2, unlike an unblazed Bragg grating, which typically includes impressed index changes that are substantially perpendicular to the longitudinal axis of the fiber core 115 of the optical fiber 105, the blazed Bragg grating 330 includes obliquely impressed index changes that are at a nonperpendicular angle to the longitudinal axis of the optical fiber 105. As mentioned above, a standard unblazed FBG partially or substantially fully reflects optical energy of a specific wavelength traveling down the axis of the fiber core 115 of optical fiber 105 back up the same axis. Blazed FBG 330 reflects this optical energy away from the longitudinal axis of the optical fiber 105. For a particular combination of blaze angle and optical wavelength, the optical energy will leave blazed FBG 330 substantially normal (i.e., perpendicular) to the longitudinal axis of the optical fiber 105. In the illustrative example of FIG. 4, an optically absorptive photoacoustic material 335 (also referred to as a “photoacoustic” material) is placed on the surface of optical fiber 105. The optically absorptive photoacoustic material 335 is positioned, with respect to the blazed grating 330, so as to receive the optical energy leaving the blazed grating. The received optical energy is converted in the optically absorptive material 335 to heat that expands the optically absorptive photoacoustic material 335. The optically absorptive photoacoustic material 335 is selected to expand and contract quickly enough to create and transmit an ultrasound or other acoustic wave that is used for acoustic imaging of the region of interest.

FIG. 4 is a cross-sectional schematic diagram illustrating generally one example of the operation of photoacoustic transducer 325 using a blazed Bragg grating 330. Optical energy of a specific wavelength, λ1, travels down the fiber core 115 of optical fiber 105 and is reflected out of the optical fiber 105 by blazed grating 330. The outwardly reflected optical energy impinges on the photoacoustic material 335. The photoacoustic material 335 then generates a responsive acoustic impulse that radiates away from the photoacoustic material 335 toward nearby biological or other material to be imaged. Acoustic energy of a specific frequency is generated by optically irradiating the photoacoustic material 335 at a pulse rate equal to the desired acoustic frequency.

In another example, the photoacoustic material 335 has a thickness 340 (in the direction in which optical energy is received from blazed Bragg grating 330) that is selected to increase the efficiency of emission of acoustic energy. In one example, thickness 340 is selected to be about ¼ the acoustic wavelength of the material at the desired acoustic transmission/reception frequency. This improves the generation of acoustic energy by the photoacoustic material.

In yet a further example, the photoacoustic material is of a thickness 300 that is about ¼ the acoustic wavelength of the material at the desired acoustic transmission/reception frequency, and the corresponding glass-based optical fiber sensing region resonant thickness 300 is about ½ the acoustic wavelength of that material at the desired acoustic transmission/reception frequency. This further improves the generation of acoustic energy by the photoacoustic material and reception of the acoustic energy by the optical fiber sensing region.

In one example of operation, light reflected from the blazed grating excites the photoacoustic material in such a way that the optical energy is efficiently converted to substantially the same acoustic frequency for which the FBG sensor is designed. The blazed FBG and photoacoustic material, in conjunction with the aforementioned FBG sensor, provide both a transmit transducer and a receive sensor, which are harmonized to create an efficient unified optical-to-acoustic-to-optical transmit/receive device. In one example, the optical wavelength for sensing is different from that used for transmission. In a further example, the optical transmit/receive frequencies are sufficiently different that the reception is not adversely affected by the transmission, and vice-versa.

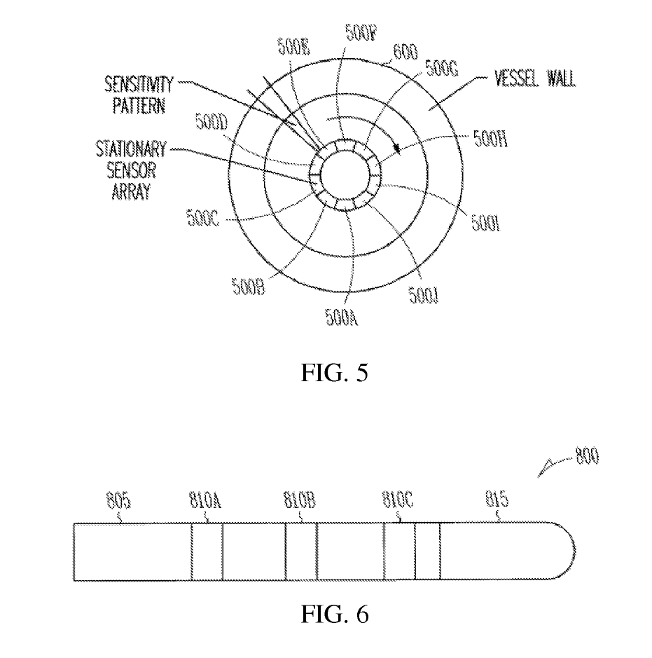

FIG. 5 is a schematic diagram illustrating generally one technique of generating an image of biological material and a vessel wall 600 through an opening in a device. In that technique, phased array images are created using a substantially stationary (i.e., non-rotating) set of multiple FBG sensors, such as FBG sensors 500A-J. FIG. 5 is a schematic diagram that illustrates generally one such phased array example, in which the signal to/from each array transducer 500A-J is combined with the signals from one or more other transducers 500A-J to synthesize a radial image line. In this example, other image lines are similarly synthesized from the array signals, such as by using specific changes in the signal processing used to combine these signals.

FIG. 6 is a schematic diagram that illustrates generally an example of a side view of a distal portion 800 of an elongate device 805. In this example, the distal portion 800 of the device 805 includes one or more openings 810A, 810B, . . . , 810N located slightly or considerably proximal to a distal tip 815 of the device 805. Each opening 810 includes one or more optical-to-acoustic transducers 325 and a corresponding one or more separate or integrated acoustic-to-optical FBG sensors 100. In one example, each opening 810 includes an array of blazed FBG optical-to-acoustic and acoustic-to-optical combined transducers 500 (such as illustrated in FIG. 5) located slightly proximal to distal tip 815 of device 805 having mechanical properties that allow the device 805 to be guided through a vascular or other lumen.

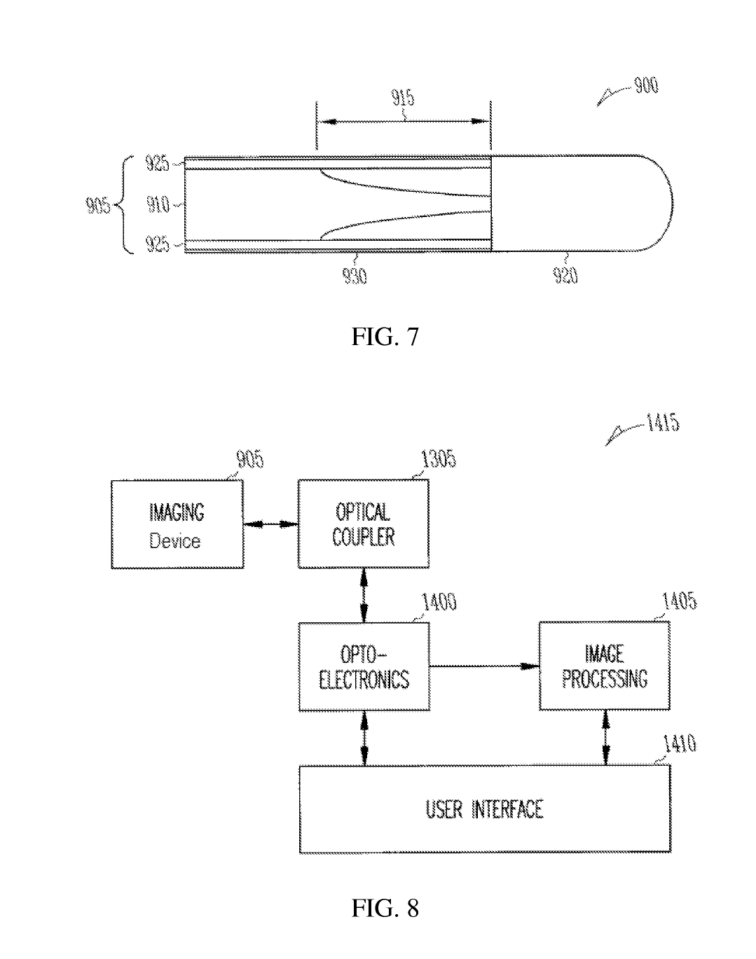

FIG. 7 is a schematic diagram that illustrates generally one example of a cross-sectional side view of a distal portion 900 of another device 905. In this example, optical fibers 925 are distributed around the device 905. In this example, the optical fibers 925 are at least partially embedded in a polymer matrix or other binder material that bonds the optical fibers 925 to the device 905. The binder material may also contribute to the torsion response of the resulting device 905. In one example, the optical fibers 925 and binder material is overcoated with a polymer or other coating 930, such as for providing abrasion resistance, optical fiber protection, and/or friction control.

In one example, before the acoustic transducer(s) is fabricated, the device 905 is assembled, such as by binding the optical fibers 925 to the device 905, and optionally coating the device 905. The opto-acoustic transducer(s) are then integrated into the core wire, such as by grinding one or more grooves in the device wall at locations of the opto-acoustic transducer window 810. In a further example, the depth of these groove(s) in the optical fiber(s) 925 defines the resonant structure(s) of the opto-acoustic transducer(s).

After the opto-acoustic transducer windows 810 have been defined, the FBGs added to one or more portions of the optical fiber 925 within such windows 810. In one example, the FBGs are created using an optical process in which the portion of the optical fiber 925 is exposed to a carefully controlled pattern of UV radiation that defines the Bragg gratings. Then, a photoacoustic material is deposited or otherwise added in the transducer windows 810 over respective Bragg gratings. One example of a suitable photoacoustic material is pigmented polydimethylsiloxane (PDMS), such as a mixture of PDMS, carbon black, and toluene.

FIG. 8 is a block diagram illustrating generally one example of the imaging device 905 and associated interface components. The block diagram of FIG. 8 includes the imaging device 905 that is coupled by optical coupler 1305 to an optoelectronics module 1400. The optoelectronics module 1400 is coupled to an image processing module 1405 and a user interface 1410 that includes a display providing a viewable still and/or video image of the imaging region near one or more acoustic-to-optical transducers using the acoustically-modulated optical signal received therefrom. In one example, the system 1415 illustrated in the block diagram of FIG. 8 uses an image processing module 1405 and a user interface 1410 that are substantially similar to existing acoustic imaging systems.

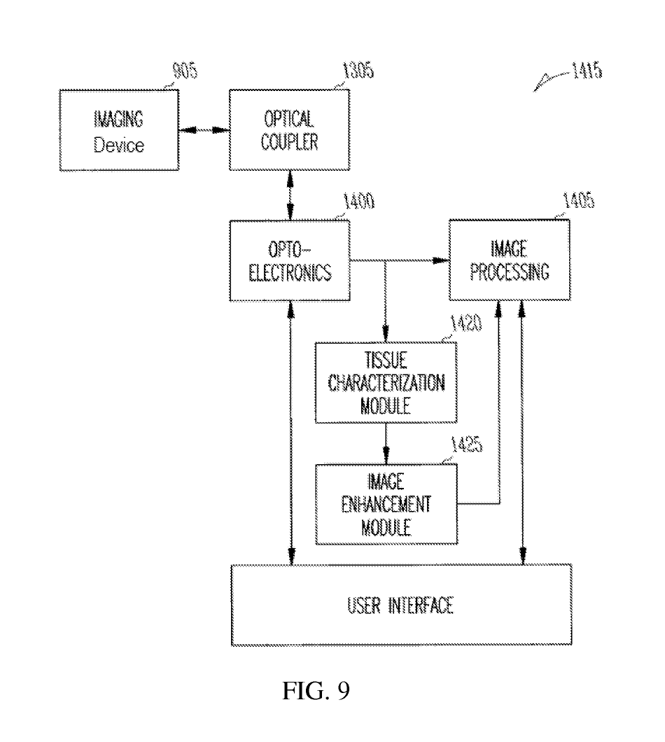

FIG. 9 is a block diagram illustrating generally another example of the imaging device 905 and associated interface components. In this example, the associated interface components include a tissue (and plaque) characterization module 1420 and an image enhancement module 1425. In this example, an input of tissue characterization module 1420 is coupled to an output from optoelectronics module 1400. An output of tissue characterization module 1420 is coupled to at least one of user interface 1410 or an input of image enhancement module 1425. An output of image enhancement module 1425 is coupled to user interface 1410, such as through image processing module 1405.

In this example, tissue characterization module 1420 processes a signal output from optoelectronics module 1400. In one example, such signal processing assists in distinguishing plaque from nearby vascular tissue. Such plaque can be conceptualized as including, among other things, cholesterol, thrombus, and loose connective tissue that build up within a blood vessel wall. Calcified plaque typically reflects ultrasound better than the nearby vascular tissue, which results in high amplitude echoes. Soft plaques, on the other hand, produce weaker and more texturally homogeneous echoes. These and other differences distinguishing between plaque deposits and nearby vascular tissue are detected using tissue characterization signal processing techniques.

For example, such tissue characterization signal processing may include performing a spectral analysis that examines the energy of the returned ultrasound signal at various frequencies. A plaque deposit will typically have a different spectral signature than nearby vascular tissue without such plaque, allowing discrimination therebetween. Such signal processing may additionally or alternatively include statistical processing (e.g., averaging, filtering, or the like) of the returned ultrasound signal in the time domain. Other signal processing techniques known in the art of tissue characterization may also be applied. In one example, the spatial distribution of the processed returned ultrasound signal is provided to image enhancement module 1425, which provides resulting image enhancement information to image processing module 1405. In this manner, image enhancement module 1425 provides information to user interface 1410 that results in a displaying plaque deposits in a visually different manner (e.g., by assigning plaque deposits a discernable color on the image) than other portions of the image. Other image enhancement techniques known in the art of imaging may also be applied. In a further example, similar techniques are used for discriminating between vulnerable plaque and other plaque, and enhancing the displayed image provides a visual indicator assisting the user in discriminating between vulnerable and other plaque.

The opto-electronics module 1400 may include one or more lasers and fiber optic elements. In one example, such as where different transmit and receive wavelengths are used, a first laser is used for providing light to the imaging device 905 for the transmitted ultrasound, and a separate second laser is used for providing light to the imaging device 905 for being modulated by the received ultrasound. In this example, a fiber optic multiplexer couples each channel (associated with a particular one of the optical fibers 925) to the transmit and receive lasers and associated optics. This reduces system complexity and costs.

In one example, the sharing of transmit and receive components by multiple guidewire channels is possible at least in part because the acoustic image is acquired over a relatively short distance (e.g., millimeters). The speed of ultrasound in a human or animal body is slow enough to allow for a large number of transmit/receive cycles to be performed during the time period of one image frame. For example, at an image depth (range) of about 2 cm, it will take ultrasonic energy approximately 26 microseconds to travel from the sensor to the range limit, and back. In one such example, therefore, an about 30 microseconds transmit/receive (T/R) cycle is used. In the approximately 30 milliseconds allotted to a single image frame, up to 1,000 T/R cycles can be carried out. In one example, such a large number of T/R cycles per frame allows the system to operate as a phased array even though each sensor is accessed in sequence. Such sequential access of the photoacoustic sensors in the guidewire permits (but does not require) the use of one set of T/R opto-electronics in conjunction with a sequentially operated optical multiplexer. In one example, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures, such as lesions, with respect to other anatomy.

In one example, in which the imaging device 905 includes 64 sequentially-accessed optical fibers having up to 10 photoacoustic transducer windows per optical fiber, 64×10=640 T/R cycles are used to collect the image information from all the openings for one image frame. This is well within the allotted 1,000 such cycles for a range of 2 cm, as discussed above. Thus, such an embodiment allows substantially simultaneous images to be obtained from all 10 openings at of each optical fiber at video rates (e.g., at about 30 frames per second for each transducer window). This allows real-time volumetric data acquisition, which offers a distinct advantage over other imaging techniques. Among other things, such real-time volumetric data acquisition allows real-time 3-D vascular imaging, including visualization of the topology of a blood vessel wall, the extent and precise location of plaque deposits, and, therefore, the ability to identify vulnerable plaque.

In certain embodiments, imaging devices are configured for imaging the tissue surrounding the esophagus. The esophagus is about 24 mm in diameter. To position the imaging sensors of the invention close enough to the walls of the esophagus so that a high resolution image may be obtained, the core wire has a diameter from about 18 mm to about 24 mm. Although not required, the core wire will typically be surrounded by optical fibers, each fiber having an optical-acoustic sensor. With a diameter of about 18 mm to about 24 mm, anywhere from 64 to 96 optical fibers are used to surround the core wire.

In other embodiments, imaging devices are configured for imaging the tissue of the gastrointestinal system. The small is about 3-4 cm in diameter. To position the imaging sensors of the invention close enough to the walls of the small intestine so that a high resolution image may be obtained, the core wire has a diameter from about 2 cm to about 4 cm. Although not required, the core wire will typically be surrounded by optical fibers, each fiber having an optical-acoustic sensor. With a diameter of about 2 cm to about 4 cm, anywhere from 128 to 512 optical fibers are used to surround the core wire.

The transverse diameter of the colon varies greatly. The cecum generally has the greatest diameter, which is usually about 9 cm in normal individuals. The transverse colon is usually about 6 cm in diameter, and the descending colon and sigmoid colon are usually about 4-5 cm in diameter. To position the imaging sensors of the invention close enough to the walls of the cecum so that a high resolution image may be obtained, the core wire has a diameter from about 7 cm to about 9 cm. Although not required, the core wire will typically be surrounded by optical fibers, each fiber having an optical-acoustic sensor. With a diameter of about 7 cm to about 9 cm, anywhere from 256 to 1000 optical fibers are used to surround the core wire. To position the imaging sensors of the invention close enough to the walls of the transverse colon so that a high resolution image may be obtained, the core wire has a diameter from about 4 cm to about 6 cm. Although not required, the core wire will typically be surrounded by optical fibers, each fiber having an optical-acoustic sensor. With a diameter of about 4 cm to about 6 cm, anywhere from 128 to 600 optical fibers are used to surround the core wire. To position the imaging sensors of the invention close enough to the walls of the descending colon or sigmoid colon so that a high resolution image may be obtained, the core wire has a diameter from about 2 cm to about 5 cm. Although not required, the core wire will typically be surrounded by optical fibers, each fiber having an optical-acoustic sensor. With a diameter of about 2 cm to about 5 cm, anywhere from 128 to 600 optical fibers are used to surround the core wire.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. An imaging device, the device comprising a core wire and more than 32 optical fibers coupled to the core wire.

2. The device according to claim 1, wherein the optical fibers completely surround the core wire.

3. The device according to claim 2, wherein a plurality of the fibers comprise an opto-acoustic sensor.

4. The device according to claim 3, wherein the opto-acoustic sensor comprises:
a blazed fiber Bragg grating within the optical fiber;
a light source that transmits light through the optical fiber; and
a photoacoustic transducer material positioned so that it receives light diffracted by the blazed fiber Bragg grating and emits ultrasonic imaging energy.

5. The device according to claim 1, wherein the core wire is about 18 mm to about 24 mm in diameter.

6. The device according to claim 1, wherein the core wire is about 2 cm to about 10 cm in diameter.

7. The device according to claim 1, wherein the number of optical fibers is selected from the group consisting of: 64 fibers, 96 fibers, and 128 fibers.

8. The device according to claim 1, wherein the fibers are embedded in the core wire.

9. The device according to claim 1, wherein the device is operably coupled to a user interface.

10. The device according to claim 9, wherein the interface comprises a tissue characterization module.

11. A method for analyzing tissue surrounding a lumen, the method comprising:
providing an imaging device, the device comprising a core wire and more than 32 optical fibers coupled to the core wire;
inserting the device into a body lumen;
using the device to image tissue that surrounds the body lumen; and
analyzing an image of the tissue, thereby analyzing the tissue surrounding the lumen.

12. The method according to claim 11, wherein the optical fibers completely surround the core wire.

13. The method according to claim 12, wherein each fiber comprises an opto-acoustic sensor.

14. The method according to claim 13, wherein the opto-acoustic sensor comprises:
a blazed fiber Bragg grating within the optical fiber;
a light source that transmits light through the optical fiber; and
a photoacoustic transducer material positioned so that it receives light diffracted by the blazed fiber Bragg grating and emits ultrasonic imaging energy.

15. The method according to claim 11, wherein the core wire is about 18 mm to about 24 mm in diameter.

16. The method according to claim 11, wherein the core wire is about 2 cm to about 10 cm in diameter.

17. The method according to claim 11, wherein the number of optical fibers is selected from the group consisting of: 64 fibers, 96 fibers, and 128 fibers.

18. The method according to claim 11, wherein the fibers are embedded in the core wire.

19. The method according to claim 11, wherein the method is operably coupled to a user interface.

20. The method according to claim 19, wherein the interface comprises a tissue characterization module.
